(19) United States
(12) Patent Application Publication
(10) Pub. No.: US 2014/0288440 A1
(43) Pub. Date: Sep. 25, 2014

(54) SYSTEMS AND METHODS FOR QUANTITATIVE CAPNOGRAM ANALYSIS

(71) Applicants: MASSACHUSETTS INSTITUTE OF TECHNOLOGY, Cambridge, MA (US); CHILDREN'S MEDICAL CENTER CORPORATION, Boston, MA (US)

(72) Inventors: Rebecca J. Asher, Cambridge, MA (US); Thomas Heldt, Cambridge, MA (US); Baruch Shlomo Krauss, Brookline, MA (US); George Cheeran Verghese, Newton, MA (US)

(73) Assignees: CHILDREN'S MEDICAL CENTER CORPORATION, Boston, MA (US); MASSACHUSETTS INSTITUTE OF TECHNOLOGY, Cambridge, MA (US)

(21) Appl. No.: 13/849,284

(22) Filed: Mar. 22, 2013

(51) Int. Cl. A61B 5/00; A61B 5/0205

(52) U.S. Cl. CPC A61B 5/7267; A61B 5/7282; A61B 5/0205; A61B 5/742; A61B 5/7246; A61B 5/7275; A61B 5/0836
USPC 600/484; 600/531

(57) ABSTRACT

Systems and methods are disclosed herein for quantitatively identifying a patient's physiological state based on one or more capnograms. One or more capnograms are acquired, each capnogram being associated with a patient and including one or more respiratory cycles, and one or more features from the one or more respiratory cycles are extracted. One or more classifiers are provided based on the one or more extracted features, and each classifier is used to select a physiological state from one or more candidate physiological states for each of the one or more respiratory cycles. For each of the selected physiological states, a likelihood value is determined, and a physiological state of the patient is determined based on the likelihood values.

### TECHNICAL FIELD

In general, this disclosure relates to quantitative capnogram analysis, and in particular, to systems and methods for disease classification using capnograms.

### BACKGROUND

Capnography refers to the non-invasive measurement of the concentration of carbon dioxide exhaled in the breath. Carbon dioxide is a byproduct of tissue metabolism. Its concentration in exhaled breath, [CO₂], can be measured noninvasively as a function of time or of volume. These processes are respectively called time-based and volumetric capnography. Capnography monitors can be found in every operating room and intensive care unit, as monitoring [CO₂] in patients is an essential aspect of modern anesthesia and respiratory care. With the advent of more portable devices, capnography can now be used in ambulatory settings as well. The waveform produced during capnography is called a capnogram and contains much information about underlying respiratory dynamics. However, current methods for assessing the capnogram are based on subjective and qualitative pattern recognition, with the clinician observing the capnogram to see if it appears roughly normal or abnormal.

In particular, present methods of inspecting the capnogram are not quantitative in nature and result in an underutilization of the capabilities of capnographic monitoring. These approaches are limited as they are dependent on the clinician's acumen and experience in visually distinguishing subtle waveform features, and therefore cannot be transferred from one clinical setting to another.

### SUMMARY

Systems and methods are disclosed herein for quantitatively identifying a patient's physiological state based on one or more capnograms. One or more capnograms are acquired, each capnogram being associated with a patient and including one or more respiratory cycles, and one or more features from the one or more respiratory cycles are extracted. One or more classifiers are provided based on the one or more extracted features, and each classifier is used to select a physiological state from one or more candidate physiological states for each of the one or more respiratory cycles. For each of the selected physiological states, a likelihood value is determined, and a physiological state of the patient is determined based on the likelihood values.

In one embodiment, the patient's physiological state pertains to a cardiorespiratory condition. Optionally, the cardiorespiratory condition includes one or more of congestive heart failure, asthma, bronchiolitis, cystic fibrosis, bronchopulmonary dysplasia, chronic obstructive pulmonary disease, or normal.

In one embodiment, the acquiring includes detecting one or more of a time of an onset of a relevant segment of a respiratory cycle, an amplitude of an onset of a relevant segment of a respiratory cycle, a time of an end of a relevant segment of a respiratory cycle, and an amplitude of an end of a relevant segment of a respiratory cycle.

In one embodiment, the acquiring includes segmenting a capnogram into a plurality of respiratory cycles.

In one embodiment, the acquiring includes assessing a signal quality of a capnogram.

In one embodiment, the acquiring includes constructing a template of a representative respiratory cycle for one or more respiratory cycles in a capnogram. Optionally, constructing the template includes computing one or more of a mean, median, standard deviation, or interquartile range at selected time points during the respiratory cycle. Optionally, an outlier portion of the capnogram is identified based on the template.

In one embodiment, extracting the one or more features includes fitting a portion of the capnogram to a parameterized function.

In one embodiment, the one or more feature values are based on physiologically significant parameters. Optionally, a feature value is selected from the group consisting of a duration of exhalation, a duration of the respiratory cycle, a signal amplitude at the end of exhalation, a slope at the beginning of exhalation, a measure of curvature at the beginning of exhalation, a measure of curvature at an intermediate point of exhalation, a measure of curvature at the end of exhalation, and a slope at the end of exhalation.

In one embodiment, the one or more classifiers are trained using a machine learning method. Optionally, the machine learning method includes discriminant analysis. Optionally, the machine learning method is based on a support vector machine.

In one embodiment, selecting a physiological state is performed by voting. Optionally, the voting is performed in one or more voting stages. Optionally, a voting stage includes selection by each classifier of a candidate physiological state for one or more respiratory cycles.

In one embodiment, determining the likelihood value of each selected physiological state includes aggregating the selections of the one or more classifiers.

In one embodiment, identifying the patient's physiological state includes comparing the likelihood values. Optionally, comparing the likelihood values is based on a predetermined threshold.

According to another aspect, the disclosure relates to a system to carry out the method described above. In particular, a system is described for quantitatively identifying a patient's physiological state based on one or more capnograms. The system comprises a processor configured to acquire one or more capnograms, each capnogram being associated with a patient and including one or more respiratory cycles. The processor is further configured to extract one or more features from the one or more respiratory cycles, provide one or more classifiers based on the one or more extracted features, and select, with each classifier, a physiological state from one or more candidate physiological states for each of the one or more respiratory cycles. The processor determines a likelihood value for each of the selected physiological states and identifies a physiological state of the patient based on the likelihood values.

In one embodiment, the patient's physiological state pertains to a cardiorespiratory condition. Optionally, the cardiorespiratory condition includes one or more of congestive heart failure, asthma, bronchiolitis, cystic fibrosis, bronchopulmonary dysplasia, chronic obstructive pulmonary disease, or normal.

In one embodiment, the acquiring includes detecting one or more of a time of an onset of a relevant segment of a respiratory cycle, an amplitude of an onset of a relevant segment of a respiratory cycle, a time of an end of a relevant segment of a respiratory cycle, and an amplitude of an end of a relevant segment of a respiratory cycle.

In one embodiment, the acquiring includes segmenting a capnogram into a plurality of respiratory cycles.

In one embodiment, the acquiring includes assessing a signal quality of a capnogram.

In one embodiment, the acquiring includes constructing a template of a representative respiratory cycle for one or more respiratory cycles in a capnogram. Optionally, constructing the template includes computing one or more of a mean, median, standard deviation, or interquartile range at selected time points during the respiratory cycle. Optionally, the processor is further configured to identify an outlier portion of the capnogram based on the template.

In one embodiment, extracting the one or more features includes fitting a portion of the capnogram to a parameterized function.

In one embodiment, the one or more feature values are based on physiologically significant parameters. Optionally, a feature value is selected from the group consisting of a duration of exhalation, a duration of the respiratory cycle, a signal amplitude at the end of exhalation, a slope at the beginning of exhalation, a measure of curvature at the beginning of exhalation, a measure of curvature at an intermediate point of exhalation, a measure of curvature at the end of exhalation, and a slope at the end of exhalation.

In one embodiment, the one or more classifiers are trained using a machine learning method. Optionally, the machine learning method includes discriminant analysis. Optionally, the machine learning method is based on a support vector machine.

In one embodiment, selecting a physiological state is performed by voting. Optionally, the voting is performed in one or more voting stages. Optionally, a voting stage includes selection by each classifier of a candidate physiological state for one or more respiratory cycles.

In one embodiment, determining the likelihood value of each selected physiological state includes aggregating the selections of the one or more classifiers.

In one embodiment, identifying the patient's physiological state includes comparing the likelihood values. Optionally, comparing the likelihood values is based on a predetermined threshold.

In one embodiment, the processor is further configured to display the capnogram and the classification results. Optionally, the processor performs in real time one or more of the acquiring, extracting, providing, selecting, determining, identifying, and displaying.

In one embodiment, the processor displays one or more of the recorded capnogram, related capnogram parameters, the computationally processed capnogram, a representative template, the classification results, and the likelihood values. Optionally, the related capnogram parameters are one or more of end-tidal carbon dioxide level, exhalation duration, and respiratory rate. Optionally, the processor displays one or more of a highlighted or color-coded segment of a respiratory cycle, an identification of an outlier cycle, and an identification of intervals of poor signal quality. Optionally, the processor displays one or more respiratory cycles superposed on the template.

In one embodiment, the processor displays the likelihood values by a representation of the classifier votes. Optionally, the representation of the classifier votes is a histogram. Optionally, the representation of the classifier votes is a color-coded set of respiratory cycles.

According to another aspect, the disclosure relates to a system for determining a physiological state associated with a capnogram. The system comprises a receiver configured to receive a plurality of capnograms, wherein each capnogram includes a plurality of respiratory cycles and is associated with a patient and one or more feature values. The system further comprises a processor configured to separate the plurality of capnograms into a training set and a testing set, wherein each capnogram in the training and testing sets is associated with a physiological state. The processor is further configured to generate a plurality of classifiers, wherein each classifier is based on the associated feature values and the physiological states of all or a subset of capnograms in the training set. In addition, the processor is configured to select, with each classifier, a candidate physiological state in a plurality of candidate physiological states for each respiratory cycle of all or a subset of the capnograms in the testing set, aggregating the candidate physiological states across the classifiers to generate an elected physiological state for each respiratory cycle in the selected capnograms, aggregating the elected physiological states across the respiratory cycles to determine the likelihood of a physiological state of the patient, and comparing the results with the known physiological state of the associated capnogram to evaluate the classifiers.

In one embodiment, the system further comprises a user interface for displaying the capnogram and an indication of the identified physiological state. Optionally, the user interface is updated in real time. Optionally, the user interface further displays the one or more feature values. Optionally, the display of the capnogram includes color-coded segments, an indication of an outlier portion, or a statistic associated with the capnogram.

According to another aspect, the disclosure relates to a method for determining a physiological state associated with a capnogram. The method comprises receiving a plurality of classifiers, wherein each classifier was previously generated based on associated feature values and the physiological states of a set of capnograms and receiving a capnogram including a plurality of respiratory cycles and associated with a patient. The method further comprises selecting, with each classifier, a candidate physiological state in a plurality of candidate physiological states for each respiratory cycle in the received capnogram, aggregating the candidate physiological states across the classifiers to generate an elected physiological state for each respiratory cycle in the received capnogram, and aggregating the elected physiological states across the respiratory cycles to determine the likelihood of a physiological state of the patient.

According to another aspect, the disclosure relates to a system for determining a physiological state associated with a capnogram. The system comprises a receiver configured to receive a plurality of classifiers, wherein each classifier was previously generated based on associated feature values and the physiological states of a set of capnograms. The receiver is further configured to receive a capnogram including a plurality of respiratory cycles and associated with a patient. The system further comprises a processor configured to select, with each classifier, a candidate physiological state in a plurality of candidate physiological states for each respiratory cycle in the received capnogram, aggregate the candidate physiological states across the classifiers to generate an elected physiological state for each respiratory cycle in the received capnogram, and aggregate the elected physiological states across the respiratory cycles to determine the likelihood of a physiological state of the patient.

According to another aspect, the disclosure relates to a method for monitoring a predicted physiological state associated with a capnogram of a patient. The method comprises receiving the capnogram including a plurality of respiratory cycles and selecting, with each classifier in a plurality of classifiers, a candidate physiological state in a plurality of candidate physiological states for each respiratory cycle in the received capnogram. The method further comprises aggregating the candidate physiological states across the classifiers to generate an elected physiological state for each respiratory cycle in the received capnogram and aggregating the elected physiological states across the respiratory cycles to determine the likelihood of a physiological state of the patient. The received capnogram and an indication of the likelihood of the physiological state are displayed to a user interface.

In one embodiment, the user interface includes a touch screen system in a hospital.

In one embodiment, the method further comprises receiving, over the user interface, user input selected from the group consisting of a capnogram, a patient, a plurality of respiratory cycles, a number of classifiers, a number of respiratory cycles, feature values associated with training the classifiers, the plurality of candidate physiological states, and likelihood threshold values.

According to another aspect, the disclosure relates to a system for determining a physiological state associated with a capnogram of a patient. The system comprises a receiver configured to receive the capnogram and a processor configured to select, with each classifier in a plurality of classifiers, a candidate physiological state in a plurality of candidate physiological states for each respiratory cycle in the received capnogram. The processor is further configured to aggregate the candidate physiological states across the classifiers to generate an elected physiological state for each respiratory cycle in the received capnogram and aggregate the elected physiological states across the respiratory cycles to determine the likelihood of a physiological state of the patient. The system further comprises a user interface configured to display the received capnogram and an indication of the likelihood of the physiological state.

In one embodiment, the user interface includes a touch screen system in a hospital.

In one embodiment, the user interface is further configured to receive user input selected from the group consisting of a capnogram, a patient, a plurality of respiratory cycles, a number of classifiers, a number of respiratory cycles, feature values associated with the classifiers, the plurality of candidate physiological states, and likelihood threshold values.

### BRIEF DESCRIPTION OF THE DRAWINGS

The above and other features of the present disclosure, including its nature and its various advantages, will be more apparent upon consideration of the following detailed description, taken in conjunction with the accompanying drawings in which:

FIG. 1 is a diagram of a capnogram, according to an illustrative implementation of the disclosure;

FIG. 2 is a block diagram of a classification system for determining a physiological state associated with a capnogram, according to an illustrative implementation of the disclosure;

FIG. 3 is a block diagram of a training system for training a set of classifiers on capnogram data, according to an illustrative implementation of the disclosure;

FIG. 4 is a block diagram of a testing system for testing a set of trained classifiers on capnogram data, according to an illustrative implementation of the disclosure;

FIG. 5 is a block diagram of an application system for using trained and tested classifiers to determine a physiological state associated with a capnogram, according to an illustrative implementation of the disclosure;

FIG. 6 is a block diagram of a computing device for performing any of the processes described herein, according to an illustrative implementation of the disclosure;

FIG. 7 is a flow diagram depicting a process, at the training stage, for training a set of classifiers on capnogram data, according to an illustrative implementation of the disclosure;

FIG. 8 is a flow diagram depicting a process, at the testing stage, for testing a set of trained classifiers on capnogram data, according to an illustrative implementation of the disclosure;

FIG. 9 is a flow diagram depicting a process, at the application stage, for using validated (trained and tested) classifiers to determine a physiological state associated with a capnogram and to display the results of the determination, according to an illustrative implementation of the disclosure;

FIG. 10 is a diagram of a template view of a capnogram, according to an illustrative implementation of the disclosure;

FIG. 11 is a diagram of a template view of a capnogram, including an indication of variability across respiratory cycles in the capnogram, according to an illustrative implementation of the disclosure;

FIG. 12 is a diagram of a capnogram with excluded respiratory cycles identified, according to an illustrative implementation of the disclosure;

FIG. 13 is a diagram of a capnogram, including a feature of exhalation duration, according to an illustrative implementation of the disclosure;

FIG. 14 is a diagram of a capnogram, including a feature of a terminal value on exhalation, according to an illustrative implementation of the disclosure;

FIG. 15 is a diagram of a capnogram, including a feature of a slope at an end of an exhalation, according to an illustrative implementation of the disclosure;

FIG. 16 is a diagram of aggregated results from a disease classification process, according to an illustrative implementation of the disclosure;

FIG. 17 is a diagram of a highlighted waveform view of a disease classification process, according to an illustrative implementation of the disclosure; and

FIG. 18 is a diagram of example capnograms from patients with various physiological states, according to an illustrative implementation of the disclosure.

### DETAILED DESCRIPTION

To provide an overall understanding of the systems and methods described herein, certain illustrative embodiments will now be described, including a system for disease classification using capnograms. However, it will be understood by one of ordinary skill in the art that the systems and methods described herein may be adapted and modified as is appropriate for the application being addressed and that the systems and methods described herein may be employed in other suitable applications, and that such other additions and modifications will not depart from the scope thereof. Generally, the computerized systems described herein may comprise one or more local or distributed engines, which include a processing device or devices, such as a computer, microprocessor, logic device or other device or processor that is configured with hardware, firmware, and software to carry out one or more of the computerized methods described herein.

The present disclosure provides systems and methods for providing disease classification using capnograms. Quantitative analysis of the capnogram allows capnography to be used as a diagnostic tool, and a capnography-based monitoring system that quantitatively classifies different respiratory diseases and different severity stages thereof constitutes a significant improvement in diagnostics. Several factors make capnography an attractive respiratory diagnostic tool. First, as a measure of ventilation, it accurately reflects underlying pulmonary physiology and pathophysiology. Second, capnography is an effort-independent measurement. Time-based capnography simply entails breathing normally through a nasal cannula. Unlike spirometry, the gold standard for measurement of airway obstruction, capnography does not require forced exhalation, which many children and subjects in respiratory distress are unable to perform. Third, with mathematical modeling and computational analysis, capnography provides an objective test: rather than relying on subjective qualitative observation for physiological state classification, capnography allows for a quantitative respiratory assessment.

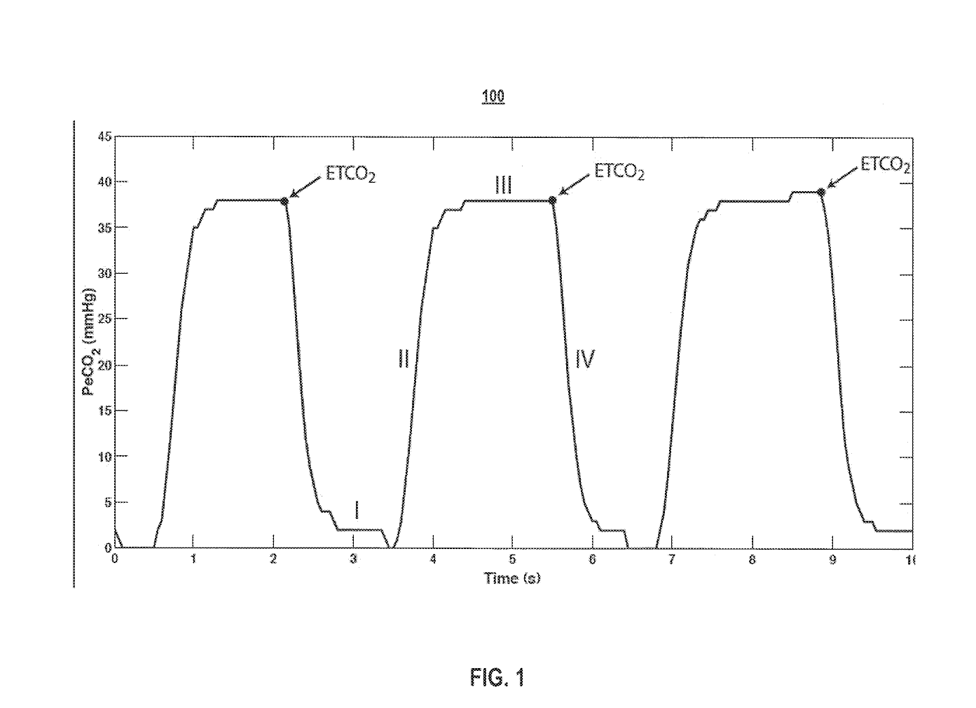

FIG. 1 is a diagram 100 of a normal time-based capnogram with four phases. Each phase of the capnogram corresponds to a specific segment of the respiratory cycle. Dead-space ventilation occurs during the first phase of exhalation, the start of alveolar gas exhalation during the second, an alveolar plateau during the third, and an inspiratory downstroke constitutes the fourth phase, to complete the waveform. Each of these phases can be estimated as a straight-line segment in the normal subject, and the terminal value of alveolar [CO₂] is defined as the End-Tidal CO₂ (ETCO₂), the maximum [CO₂] in each breath.

The capnogram contains important information about metabolic and cardiorespiratory function. The respiratory rate is calculated from the beginning of alveolar gas exhalation (the start of phase two) on one breath to the corresponding point of the next, while the amplitude of the capnogram at the end of exhalation, the end-tidal CO₂ value, reflects arterial [CO₂] and is an indicator of metabolic and cardiorespiratory function. While these two parameters are important clinically, the entire waveform contains more information than can be aggregated by these two summary statistics.

An objective of the present disclosure is to quantify the capnogram in order to discriminate among various respiratory disorders. Quantitative analyses of the capnogram are conducted by extracting several capnogram features, including physiological features, and performing classification, which may be done by discriminant analysis with voting, in an illustrative embodiment. In a first step, classifiers are trained on a set of training capnogram data for which patients' physiological states are known. A physiological state may correspond to any abnormal or normal physiological condition. For example, a physiological state may be related to cardiorespiratory condition, such as congestive heart failure, asthma, bronchiolitis, cystic fibrosis, bronchopulmonary dysplasia, chronic obstructive pulmonary disease, or normal. In a second step, the classifiers are tested on a set of testing capnogram data for their ability to distinguish records from patients with different physiological states. In a third step, the classifiers are applied to a patient capnogram for which the physiological state may be unknown. The systems and methods described herein demonstrate the diagnostic potential of capnography.

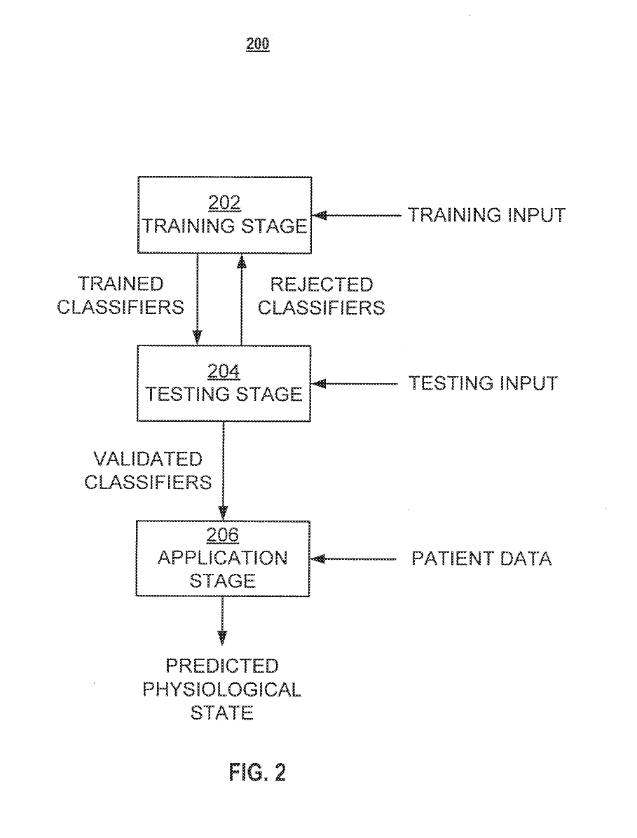

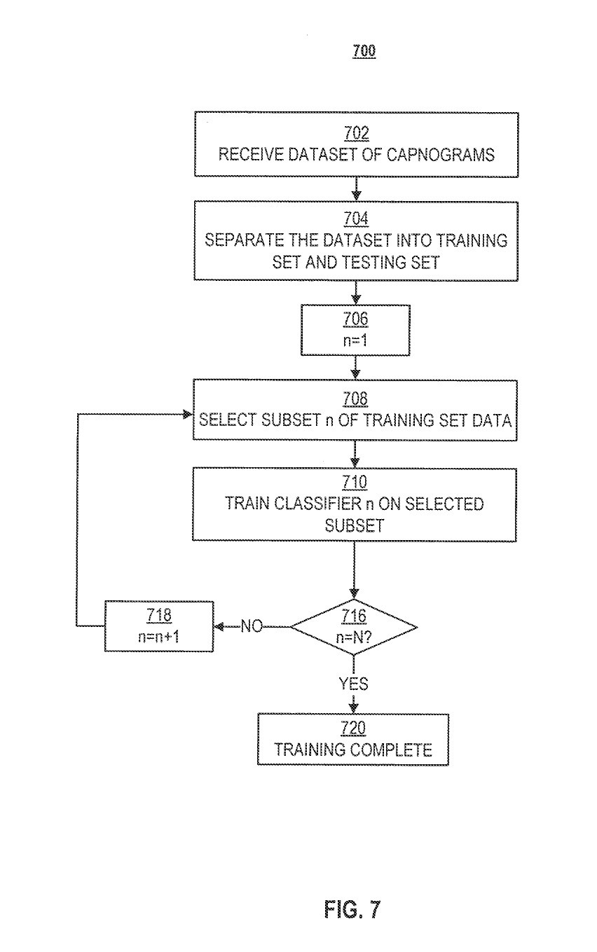

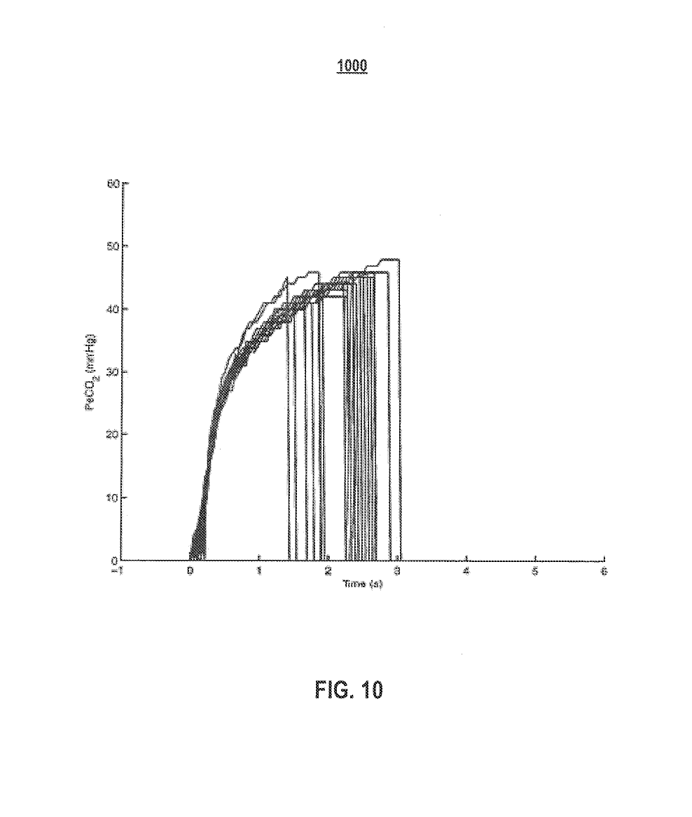

The systems and methods of the present disclosure may be described in more detail with reference to FIGS. 2-17. More particularly, an exemplary system for providing disease classification and its components are described with reference to FIGS. 2-6. The system may provide disease classification as described with reference to flow charts in FIGS. 7-9. In addition, example capnogram data including template views, identified outlier breaths, extracted features, and classification results are described with reference to FIGS. 10-17.

FIG. 2 is an illustrative block diagram of a classification system 200 for determining a physiological state associated with a capnogram. The system 200 includes a training stage 202, a testing stage 204, and an application stage 206. Inputs to the system 200 include training input data to train a set of classifiers, testing input data to test the set of trained classifiers, and data recorded from a patient. The system 200 uses the trained and tested classifiers and the patient data to provide a predicted physiological state of the patient.

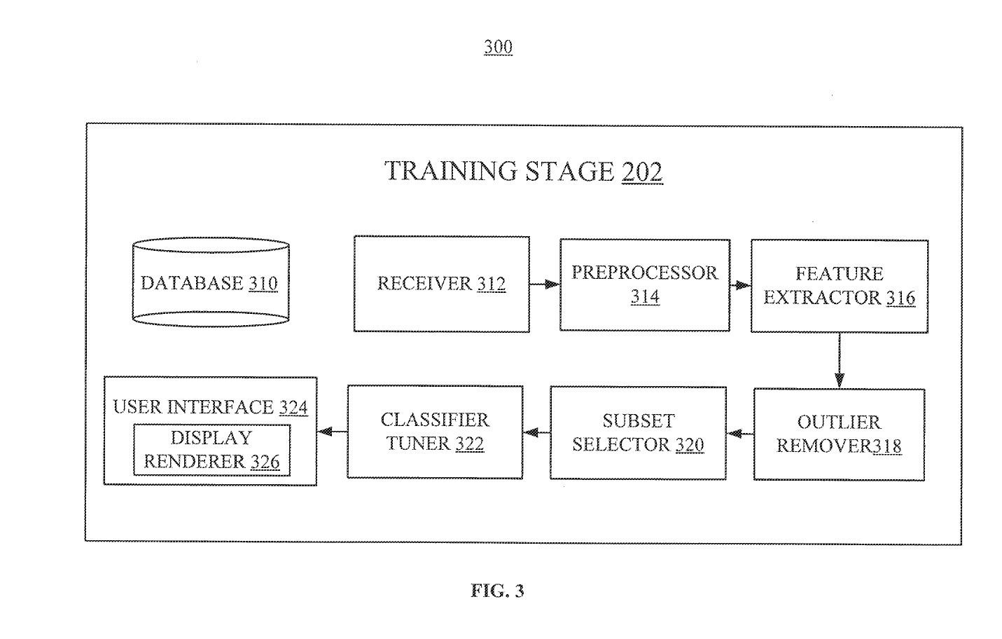

The training stage 202 receives a set of training input data and provides a set of trained classifiers to the testing stage 204. The set of training input data includes a set of training capnogram data recorded from a first group of patients and a set of the patients' physiological states. The components of the training stage 202 are described in detail in relation to FIG. 3, and the training stage 202 may operate on the training input data according to the method as described in relation to FIG. 7. In particular, the training stage 202 may select subsets of the training input data and train a classifier on each selected subset.

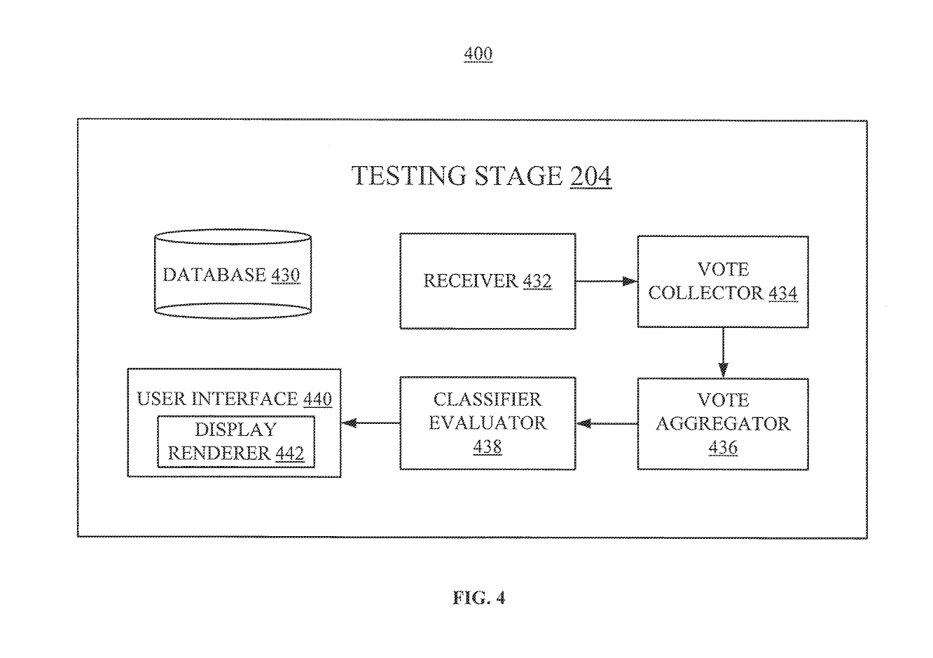

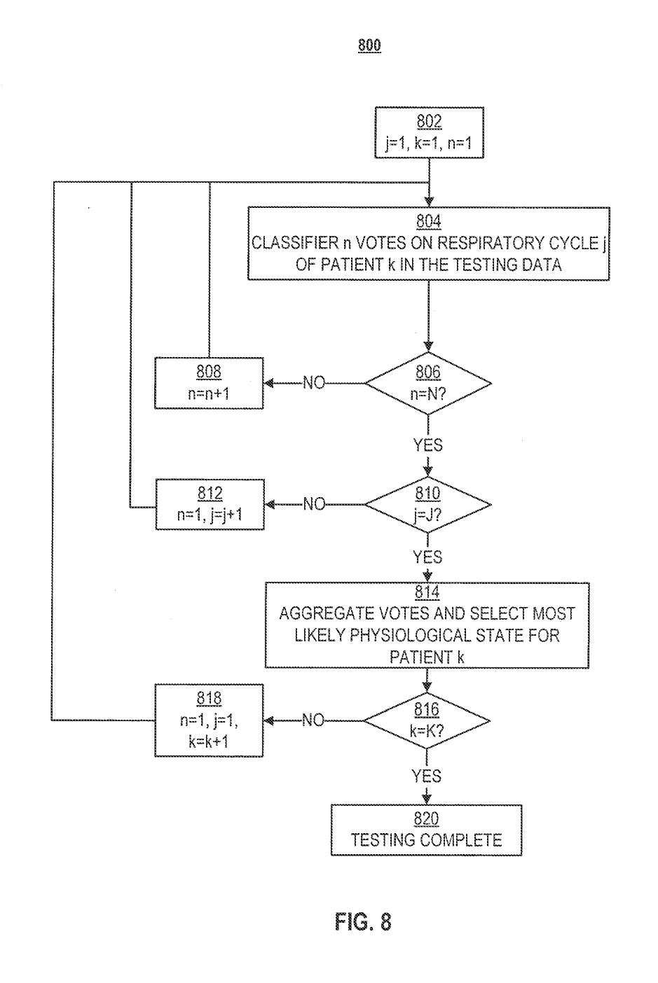

The testing stage 204 receives the set of trained classifiers from the training stage 202 and a set of testing input data. The set of testing input data includes a set of testing capnogram data recorded from a second group of patients and a set of the patients' physiological states. The components of the testing stage 204 are described in detail in relation to FIG. 4, and the testing stage 204 may operate on the testing input data and the trained classifiers according to the method as described in relation to FIG. 8. In particular, the testing stage 204 may aggregate votes from the trained classifiers operating on the testing input data and compare the physiological states predicted by the aggregated votes to the corresponding set of actual physiological states from the second group of patients. If there is a sufficient match between the predicted and actual physiological states, the testing stage 204 validates the classifiers and provides the validated classifiers to the application stage 206.

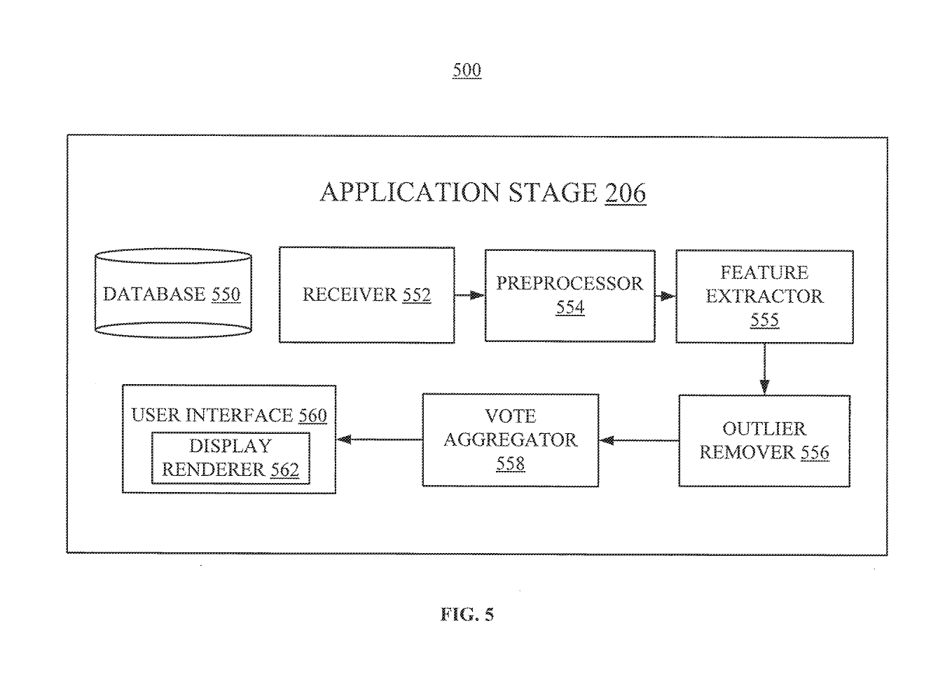

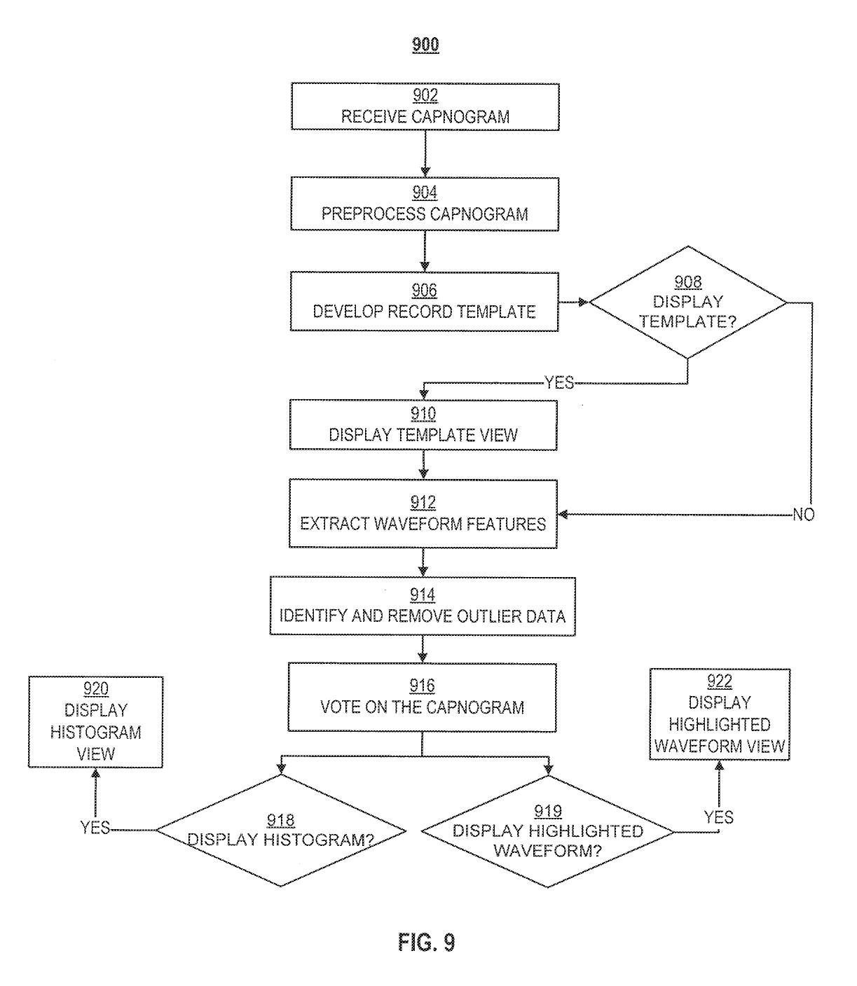

The application stage 206 receives the set of validated classifiers from the testing stage 204 and data recorded from a patient. The data may include a capnogram recorded from the data, and the physiological state of the patient may be unknown. The components of the application stage 206 are described in detail in relation to FIG. 5, and the application stage 206 may operate on the patient data and the validated classifiers according to the method as described in relation to FIG. 9. In particular, the application stage 206 may aggregate votes from the validated classifiers operating on the patient data to determine a predicted physiological state associated with the patient. The predicted physiological state may be provided by the system 200 to a user such as a medical professional.

FIG. 3 is an illustrative block diagram of a training stage 202 for training a set of classifiers on a set of training capnogram data. The training stage 202 includes several components for executing the processes described herein. In particular, the training stage 202 includes a database 310, a receiver 312, a preprocessor 314, a feature extractor 316, an outlier remover 318, a subset selector 320, a classifier tuner 322, and a user interface 324 that includes a display renderer 326. The training stage 202 may operate on training input data according to the method as described in relation to FIG. 7. The database 310 may be used to store any data related to training a set of classifiers as described herein.

The training stage 202 receives training input data over the receiver 312. The receiver 312 may provide an interface with a data source, which may transmit training capnogram data and corresponding patient physiological states to the training stage 202. The training capnogram data may be recorded from a first group of patients, and the physiological states of the first group of patients may be known and transmitted to the receiver 312. In particular, the training capnogram data may include a number of capnograms, each waveform including multiple respiratory cycles.

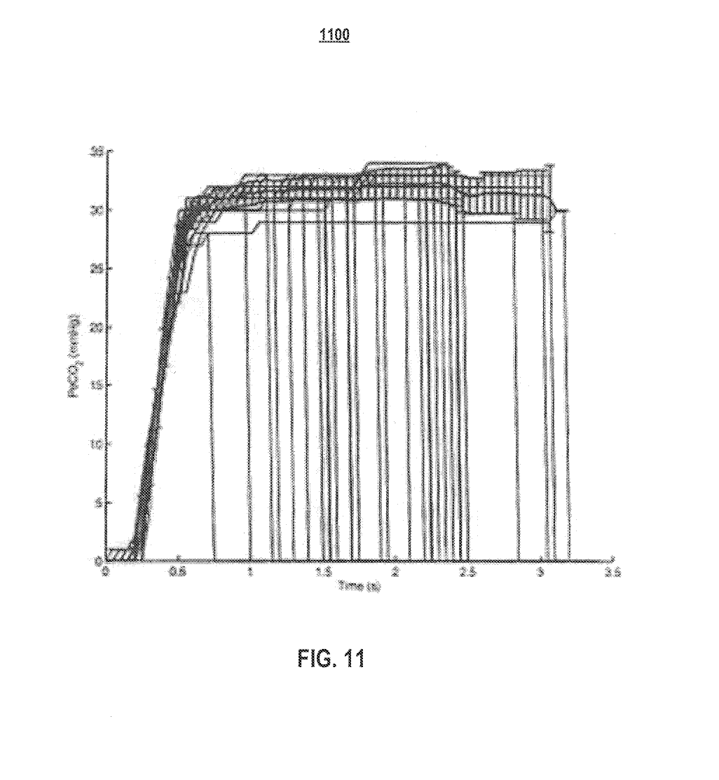

After the training data are received, the preprocessor 314 may process the training data to convert the data into a suitable form for performing analysis. For example, the preprocessor 314 may generate a template view of the received capnogram data by identifying a suitable time period representative of one respiratory cycle of a capnogram. A template view is a display of a representative respiratory cycle of a capnogram, and may be generated by overlaying different respiratory cycles from the same capnogram. Examples of template views are shown and described in relation to FIGS. 10 and 11.

Referring now to FIG. 10, a diagram 1000 shows a template view of a capnogram. Capnograms are most commonly viewed as a waveform strip on a long time-axis. Examining records that are 20-30 minutes long in this fashion can be a daunting task. To facilitate the viewing of the capnogram, a template view presents an alternative to the ordinary sequential waveform view of the capnogram signal. The exhalation portions of the respiratory cycles from a single record are overlaid over the duration of the longest exhalation in the record. To do this, the exhalations are anchored at some fixed value of PeCO₂, the partial pressure of carbon dioxide in the breath. For the template view shown in FIG. 10, 15 mmHg was used to anchor the exhalations. Thus, all exhalations cross 15 mmHg at the same time in the template view in this example. However, in general, any suitable value may be used to anchor the template.

In certain implementations, the average exhalation is computed by taking the mean PeCO₂ at every time sample in the overlaid exhalations. The average exhalation can be thought of as a composite breath that is representative of the record as a whole. Rather than paying too much attention to outlier exhalations, the composite exhalation allows for quick viewing of each record.

Referring now to FIG. 11, a diagram 1100 shows a template view of a capnogram, including an indication of variability across respiratory cycles in the capnogram. In particular, the template view in FIG. 11 is similar to the template view shown in FIG. 10, but FIG. 11 includes vertical bars indicative of the standard deviation across exhalations. The template view proves very useful in that a general, or exemplary, exhalation can be seen when all the exhalations from a single record are overlaid.

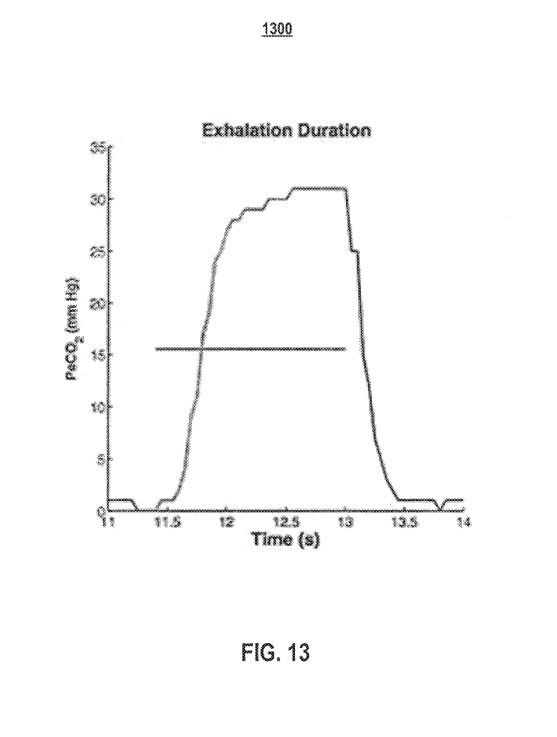

Referring back to FIG. 3, after the training data are processed by the preprocessor 314, the feature extractor 316 extracts features from the remaining validated exhalations. The features may be characteristics of the capnogram that are directly correlated to physiological processes. Features may be indicative of respiratory function, and examples of features include respiratory rate, exhalation duration, end-exhalation value, end-exhalation slope, or any other suitable feature of a capnogram. Use of feature extraction is advantageous because the features are understandable and easy to link to physiologic function. In addition, these features are straightforward to extract from the capnogram. FIGS. 13-15 show example diagrams of capnograms including different features that may be extracted by the feature extractor 316.

Referring back to FIG. 3, the outlier remover 318 may be configured to identify and remove outlier exhalations. For example, the template view of a capnogram is used as an exemplary breath. Exhalations that deviate significantly from the template are then excluded from analysis and classification. For example, an outlier exhalation may be identified if the exhalation includes a number of samples that fall outside a specified range.

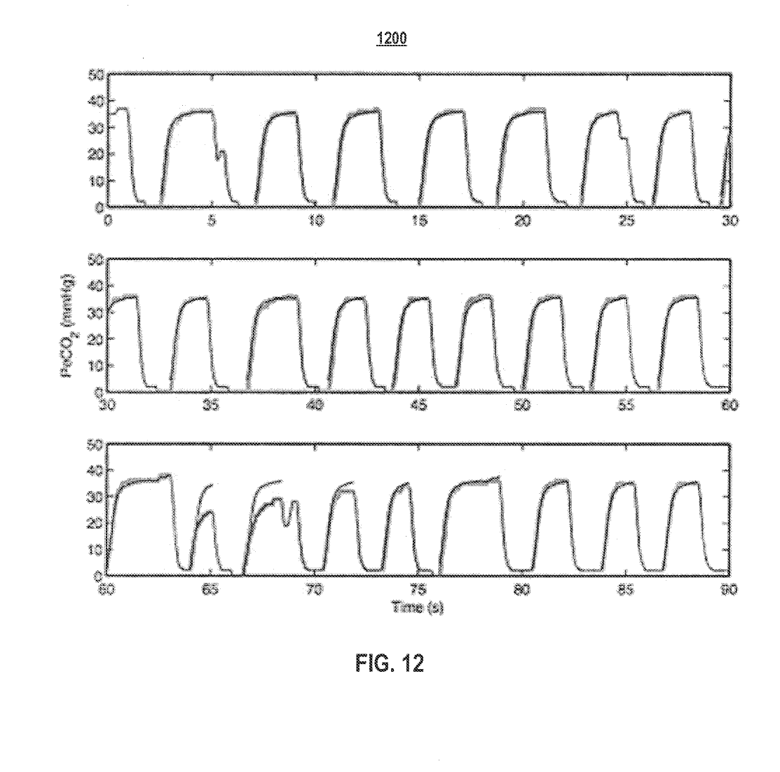

In practice, pathologic exhalations may tend to be more disordered and irregular than exhalations recorded from healthy patients, which may tend to be more consistent. Thus, outliers may tend to occur more often in pathologic records. The outlier remover 318 may identify outlier exhalations by using one or more exhalation exclusion criteria, which may include exhibiting a positive or negative deviation from the mean exhalation above a specified multiple of the standard deviation (the multiple can be different for positive and negative deviations, for example). Additionally, if an extracted feature (described in more detail below with reference to FIGS. 13-15) deviates more than a specified multiple of the standard deviation from the record mean, the outlier remover 318 may identify the exhalation as an outlier. FIG. 12 is a diagram of a capnogram recorded from a healthy patient with lines differentiating validated exhalations (i.e., exhalations identified as non-outliers) and outlier exhalations. The validated exhalations line up fairly well with one another, while the outlier exhalations deviate significantly from the other exhalations.

Referring now to FIG. 13, a diagram 1300 shows a capnogram, including a feature of exhalation duration. Exhalation duration is measured from the onset of exhalation, the time at which the capnogram slope becomes positive, until the last time at which [CO₂] is recorded on the exhalation (yielding ETCO₂).

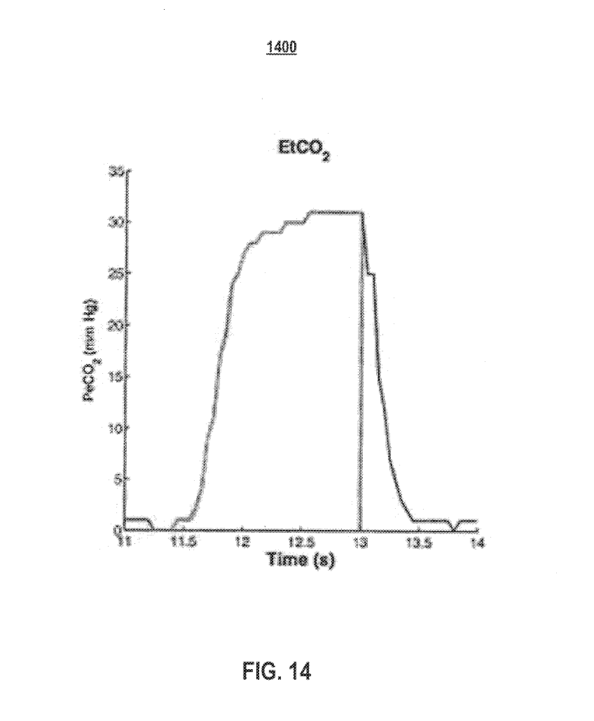

Referring now to FIG. 14, a diagram 1400 shows a capnogram, including a feature of a terminal value on exhalation. The terminal value on exhalation is captured just before the capnogram begins decreasing and is labeled as the ETCO₂ value. This quantity is highly correlated with respiratory health. Obstructive disease patients are generally seen to exhibit high ETCO₂ values, reflecting high arterial [CO₂], as they do not satisfactorily expel carbon dioxide during exhalation. On the other hand, restrictive lung disease results in decreased ETCO₂ levels since perfusion of carbon dioxide into the alveoli is impaired.

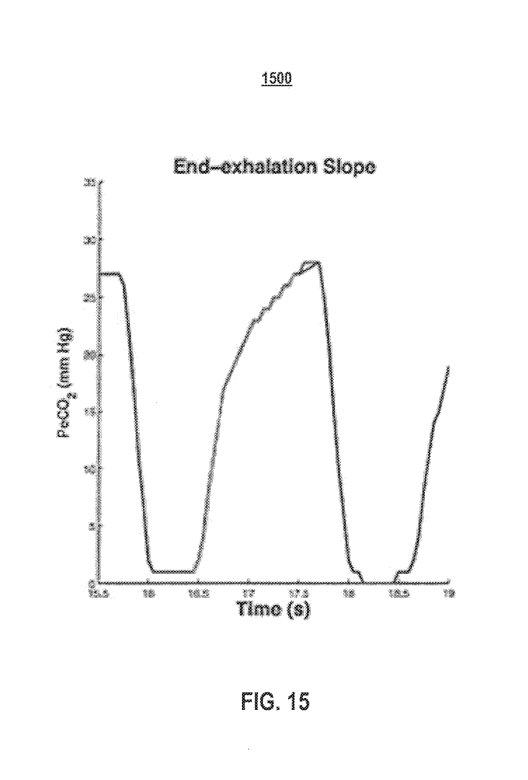

Referring now to FIG. 15, a diagram 1500 shows a capnogram, including a feature of a slope at an end of an exhalation. The end-exhalation slope represents a distinctive feature of COPD. In an example, to extract the end-exhalation slope, the feature extractor 316 implements a linear regression over the last fifth of the capnogram exhalation. The slope of this regression line is then taken as the end-exhalation slope. Because normal breathing results in a relatively flat alveolar plateau and obstructive disease yields a more rounded shape, the end-exhalation slope feature is especially useful in distinguishing obstructive from normal exhalations.

Referring back to FIG. 3, after the feature extractor 316 extracts the features from the training set of capnogram data, the subset selector 314 selects subsets of the training capnogram data. As an example, the subset selector 314 may randomly select a fixed number of the patients in the first group of patients to form a first subset of the training data. The number of selected patients may be based on user input received over the user interface 320. For example, the user input may include the number of patients to select, or the user input may include a percentage of the number of patients to select. Additional subsets may also be subsequently selected in the same way. The number of selected subsets may be based on a number of desired classifiers, and may be determined based on user input received over the user interface 320. The total number of selected subsets is N.

After various subsets of the training capnogram data are selected, the classifier tuner 322 trains a classifier on each subset to determine a decision rule for the subset. In particular, the decision rule may be based on the extracted features from the training data set and the known physiological states of the patients in the training data set. A classifier may provide a map from any set of feature values to a physiological state.

FIG. 4 is an illustrative block diagram of a testing stage 204 for testing a set of classifiers on a set of testing capnogram data. The testing stage 204 includes several components for executing the processes described herein. In particular, the testing stage 204 includes a database 430, a receiver 432, a vote collector 434, a vote aggregator 436, a classifier evaluator 438, and a user interface 440. The testing stage 204 may operate on testing input data and a set of trained classifiers according to the method described in relation to FIG. 8. The database 430 may be used to store any data related to testing a set of classifiers as described herein.

The testing stage 204 receives testing input data and a set of trained classifiers over the receiver 432. The receiver 432 may provide an interface with a data source, which may transmit testing capnogram data and corresponding patient physiological states to the testing stage 204. The testing capnogram data may be recorded from a second group of patients (i.e., which may be different from the first group of patients making up the set of testing capnogram data), and the physiological states of the second group of patients may be known and transmitted to the receiver 432. In particular, there may be K patients in the second group of patients, such that the testing capnogram data include K capnograms, each waveform including multiple respiratory cycles. The receiver 432 may also form an interface with the training stage 202 to receive a set of trained classifiers from the training stage 202. In particular, each trained classifier in the set of trained classifiers may include a decision rule based on patients' capnogram data indicating a most likely physiological state corresponding to the patient.

After the testing data and the set of classifiers are received, the vote collector 434 collects votes from the N trained classifiers based on the capnogram record from each patient in the second group of patients. The votes correspond to candidate physiological states that are selected based on the decision rule of each trained classifier. In particular, a number J of respiratory cycles may be selected (J may be determined based on user input received over the user interface 440, for example), and for each patient, the classifiers may vote on each of the J respiratory cycles. Thus, for each patient record in the set of testing capnogram data, the vote collector 434 determines N×J votes for the candidate physiological states.

After the votes have been collected, the vote aggregator 436 aggregates the votes. In certain implementations, the vote aggregator 436 combines the votes in two steps for each patient. A first step includes a “per respiratory cycle” aggregation, and a second step includes a “per patient” aggregation. The result of the two steps is a selection of a most likely physiological state of a patient in the second group of patients.

In a first step, the vote aggregator 436 performs a “per respiratory cycle” aggregation by combining the N votes across the N classifiers (i.e., one vote per classifier) for a given respiratory cycle. For example, the vote aggregator 436 may determine a most likely candidate physiological state corresponding to a respiratory cycle by determining whether the number of votes for the physiological state satisfies some criterion. For example, a threshold value may be based on user input received over the user interface 440 and may be a fixed number or a fixed percentage of the N votes. In another example, the criterion may require the vote aggregator 436 to simply select the candidate physiological state with the most votes, regardless of whether the number of votes exceeds some threshold. In another example, the N votes may be combined to obtain an average ranging from 0 to 1 that is retained in the next voting step. The vote aggregator 436 repeats this for each of the J respiratory cycles in a patient's record to provide a set of J resulting votes (i.e., one resulting vote per respiratory cycle).

In a second step, the vote aggregator 436 performs a “per patient” aggregation by combining the set of J resulting votes to determine a most likely physiological state for the patient. For example, the vote aggregator 436 may determine a most likely physiological state of the patient by determining whether the number of votes for the physiological state satisfies some criterion. The criterion may be the same or different from the “per respiratory cycle” aggregation. For example, a threshold value may be based on user input received over the user interface 440 and may be a fixed number or a fixed percentage of the J resulting votes. In another example, the criterion may require the vote aggregator 436 to simply select the candidate physiological state with the most votes, regardless of whether the number of votes exceeds some threshold. If there are K patients in the second group of patients, the vote aggregator 436 repeats this for each of the K patients to provide a set of K elected physiological states (i.e., one physiological state per patient).

After the votes are aggregated, the classifier evaluator 438 performs a validation of the classifiers. In particular, the classifier evaluator 438 compares the set of K elected physiological states to the known physiological states of the K patients to determine a level of accuracy of the classifiers. For example, the classifier evaluator 438 may determine that the classifiers are validated if the number of correctly elected physiological states that match the known physiological states exceeds a threshold. The threshold may be a fixed number or a percentage and may be provided by a user over the user interface 440. In addition, the classifier evaluator 438 may consider the variation in the votes. For example, if the votes are highly varied across classifiers, or if the vote aggregator 436 is unable to determine a most likely physiological state because none of the physiological states satisfies the required criterion, the classifier evaluator 438 may determine that the trained classifiers are invalid. If the classifier evaluator 438 determines that the trained classifiers are invalid, the testing stage 204 may provide an instruction to the training stage 202 to repeat the training process (e.g. trying a different set of features, a different number of classifiers, or a change in any other suitable parameter in the training process). For example, the testing stage 204 may return the rejected classifiers to the training stage 202. These steps may be repeated until a set of classifiers is identified that satisfies the criterion required by the classifier evaluator 438. The testing stage 204 then provides the validated set of classifiers to the application stage 206.

FIG. 5 is an illustrative block diagram of an application stage 206 for applying a set of classifiers to a patient's data to predict a physiological state of the patient. The application stage 206 includes several components for executing the processes described herein. In particular, the application stage 206 includes a database 550, a receiver 552, a preprocessor 554, a feature extractor 555, an outlier remover 556, a vote aggregator 558, and a user interface 560. The application stage 206 may operate on a patient's data and a set of validated classifiers according to the method as described in relation to FIG. 9. The database 550 may be used to store any data related to applying a set of classifiers to a patient's data as described herein.

The application stage 206 receives a patient's data and a set of validated classifiers over the receiver 552. The receiver 552 may provide an interface with a data source, which may transmit the patient's capnogram data to the application stage 206. The patient's capnogram may be recorded from a patient not included in either the first or second group of patients, and the physiological state of the patient may be unknown. In particular, the patient's capnogram data may include a number of respiratory cycles. The receiver 552 may also form an interface with the testing stage 204 to receive a set of validated classifiers from the testing stage 204. In particular, each validated classifier in the set of validated classifiers may include a decision rule based on one or several patients' capnogram data indicating a most likely physiological state corresponding to the patient.

After the patient's data and the set of validated classifiers are received, the preprocessor 554 may process the patient's data to convert the data into a suitable form for performing analysis. For example, the preprocessor 554 may generate a template view of the patient's capnogram data by identifying a suitable time period representative of one respiratory cycle of a capnogram. A template view is a display of a representative respiratory cycle of a capnogram, and may be generated by overlaying different respiratory cycles from the same capnogram. Examples of template views are shown in FIGS. 10 and 11.

The feature extractor 555 extracts features from the remaining exhalations. The features may be characteristics of the capnogram that are directly correlated to physiological processes. Features may be indicative of respiratory function, and examples of features include respiratory rate, exhalation duration, end-exhalation value, end-exhalation slope, or any other suitable feature of a capnogram. These features are described in more detail in relation to FIGS. 13-15.

In certain implementations, the average exhalation is computed by taking the mean PeCO₂ at every time sample in the overlaid exhalations. The average exhalation is a representative exhalation and may be used by the outlier remover 556 to remove outlier exhalations as described in relation to FIGS. 3 and 12. In particular, the outlier remover 556 may be configured to identify and remove outlier exhalations. For example, exhalations that deviate significantly from the template may be excluded from analysis and classification. For example, an outlier exhalation may be identified if the exhalation includes a number of samples that fall outside a specified range. In another example, an outlier exhalation may be identified if an extracted feature of the exhalation deviates more than a specified multiple of the standard deviation from the record mean.

After outlier exhalations are removed, the validated classifiers are applied to the patient's capnogram data, and the vote aggregator 558 collects and combines the votes selected by each validated classifier. As described in relation to FIG. 4, the vote aggregator 558 may aggregate the votes in two steps. A first step may include a “per respiratory cycle” aggregation, and a second step may include a “per patient” aggregation. The result of the two steps is a selection of a most likely physiological state of a patient in the second group of patients.

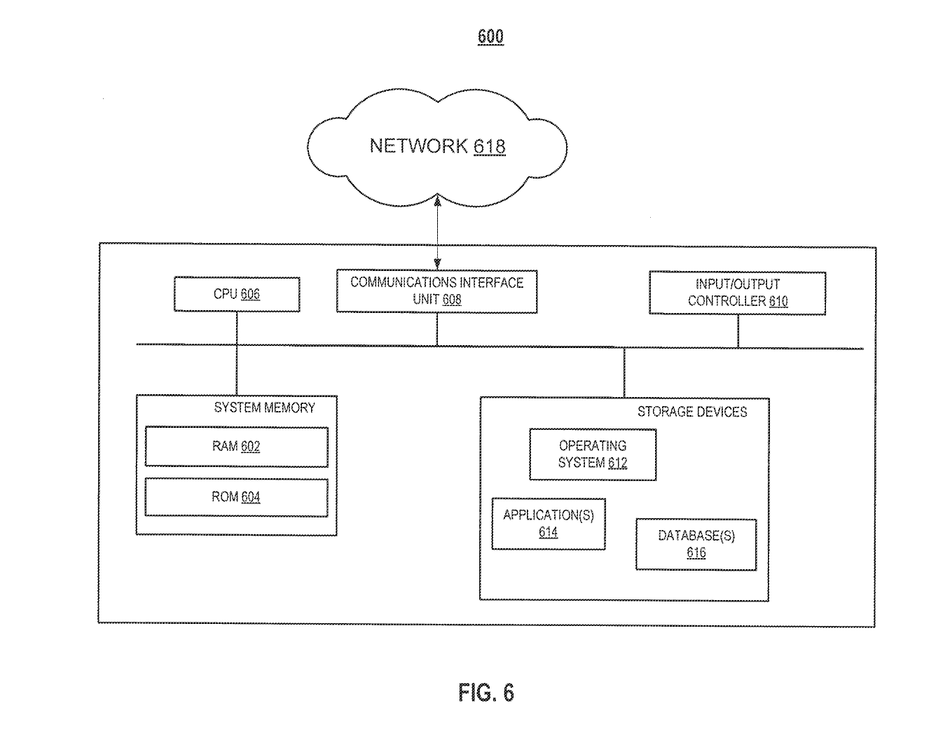

FIG. 6 is a block diagram of a computing device for performing any of the processes described herein, according to an illustrative embodiment. Each of the components of these systems may be implemented on one or more computing devices 600. In certain aspects, a plurality of the components of these systems may be included within one computing device 600. In certain implementations, a component and a storage device may be implemented across several computing devices 600.

The computing device 600 comprises at least one communications interface unit, an input/output controller 610, system memory, and one or more data storage devices. The system memory includes at least one random access memory (RAM 602) and at least one read-only memory (ROM 604). All of these elements are in communication with a central processing unit (CPU 606) to facilitate the operation of the computing device 600. The computing device 600 may be configured in many different ways. For example, the computing device 600 may be a conventional standalone computer or, alternatively, the functions of computing device 600 may be distributed across multiple computer systems and architectures. In FIG. 6, the computing device 600 is linked, via network or local network, to other servers or systems.

The computing device 600 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 608 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 606 comprises a processor, such as one or more conventional microprocessors and one or more supplementary co-processors such as math co-processors for offloading workload from the CPU 806. The CPU 606 is in communication with the communications interface unit 608 and the input/output controller 610, through which the CPU 606 communicates with other devices such as other servers, user terminals, or devices. The communications interface unit 608 and the input/output controller 610 may include multiple communication channels for simultaneous communication with, for example, other processors, servers or client terminals in the network 618.

The CPU 606 is also in communication with the data storage device. The data storage device may comprise an appropriate combination of magnetic, optical or semiconductor memory, and may include, for example, RAM 602, ROM 604, flash drive, an optical disc such as a compact disc or a hard disk or drive. The CPU 606 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 606 may be connected to the data storage device via the communications interface unit 608. The CPU 606 may be configured to perform one or more particular processing functions.

The data storage device may store, for example, (i) an operating system 612 for the computing device 600; (ii) one or more applications 614 (e.g., computer program code or a computer program product) adapted to direct the CPU 606 in accordance with the systems and methods described here, and particularly in accordance with the processes described in detail with regard to the CPU 606; or (iii) database(s) 616 adapted to store information that may be utilized to store information required by the program.

The operating system 612 and applications 614 may be stored, for example, in a compressed, an uncompiled and an encrypted format, and may include computer program code. The instructions of the program may be read into a main memory of the processor from a computer-readable medium other than the data storage device, such as from the ROM 604 or from the RAM 602. While execution of sequences of instructions in the program causes the CPU 606 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions in relation to performing classification of physiological states based on capnograms as described herein. The program also may include program elements such as an operating system 612, a database management system and “device drivers” that allow the processor to interface with computer peripheral devices (e.g., a video display, a keyboard, a computer mouse, etc.) via the input/output controller 610.

The term “computer-readable medium” as used herein refers to any non-transitory medium that provides or participates in providing instructions to the processor of the computing device 600 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 606 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 600 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

The systems shown in FIGS. 2-6 may provide disease classification as described with reference to flowcharts in FIGS. 7-9. In particular, the training stage 202 may use the method shown in FIG. 7 to train a set of classifiers on a set of training capnogram data. After the set of classifiers are trained, the testing stage may use the method shown in FIG. 8 to validate the set of trained classifiers. Finally, the application stage may use the method shown in FIG. 9 to apply the validated classifiers to a patient's capnogram data to identify a predicted physiological state of the patient.

FIG. 7 is a flowchart of a method used by the training stage 202 to train a set of classifiers on a set of training data. Classifiers are trained on a training set of capnogram data and their corresponding features. In particular, the training set of capnogram data includes a set of capnogram data from patients with corresponding known physiological states. In certain implementations, the physiological states correspond to obstructive or restrictive lung disease, including chronic obstructive pulmonary disease (COPD) and congestive heart failure (CHF). More generally, these patients may have been diagnosed with any number of diseases, such as CHF, asthma, bronchiolitis, cystic fibrosis, bronchopulmonary dysplasia, COPD, or any other disease. Alternatively, a patient may be determined to have a healthy physiological state. Depending on a patient's physiological state, a capnogram associated with the patient may exhibit different features. To train a classifier, a subset of the training set is selected (a subset of the records or patients in the training set, for example), and a classifier is generated based on the waveform features of the selected subset.

The method 700 includes the steps of receiving a dataset of capnograms (step 702), separating the dataset into a training set and a testing set (step 704), and initializing one iteration parameter n to one (step 706). A subset n of the training set data is selected (step 708), and a classifier n is trained on the selected subset (step 710). Steps 708 and 710 are repeated until the desired number of classifiers (i.e., N) have been trained

At step 702, a dataset of capnograms is received, for which patient diagnoses are known, such that information is available regarding the physiological state of the patients. At step 704, the received dataset is separated into a training set and a testing set. The training set is used to develop the classifiers and is provided as input to the training stage 202. The testing set is used to assess the performance of the resulting classifiers and is provided as input to the testing stage 204. An example method of assessing the performance of the classifiers in the testing stage 204 is described in relation to FIG. 8.

At step 706, one iteration parameter n is initialized to one. The iteration parameter n is representative of a selected subset of the training set.

At step 708, the subset selector 320 selects an n^{th} subset of the training set data. As an example, a random subset of the training set data may be selected. For example, if there are 100 patients in the training set data, capnograms from 70 patients may be randomly selected to form the subset n. Optionally, the training set data may be processed by the preprocessor 314 (i.e., to get the training set data into a suitable form), the feature extractor 316 (i.e., to extract features from the capnograms), and/or the outlier remover 318 (i.e., to remove outlier exhalations) in any order. These processes are described in more detail in relation to FIG. 3.

At step 710, the n^{th} classifier is trained on the corresponding subset. To train a classifier, a number of respiratory cycles of the patients in the n^{th} subset may be used. For example, the first 35 valid breaths of each patient may be used. Because the physiological states of the patients in the training set are known (i.e., CHF versus COPD), the n^{th} classifier is trained on the features of the patient breaths. In particular, to train a classifier, the classifier tuner 322 may define a decision rule, for which any set of extracted feature values may be mapped to a physiological state.

At decision block 716, it is determined whether the iteration parameter n equals the desired total number of subsets N. If not, the iteration parameter n is incremented at step 718 and the process returns to step 708 to select another subset of training set data.

When iteration parameter n has reached its final value, training is complete at step 720. In particular, as a result of the training, N classifiers have been generated. The classifiers may be different because they were tuned for optimal performance on different subsets of the training set records, though they all had the same mathematical/computational structure.

FIG. 8 is a flowchart of a method used by the testing stage 204 to test a set of trained classifiers on a set of testing data. The method 800 includes the steps of initializing three iteration parameters j, k, and n to one (step 802), and allowing classifier n to vote on the respiratory cycle j of a patient k in the testing data. For each patient k, this process is repeated N (number of classifiers)×J (number of respiratory cycles examined) times. When the voting process is complete for patient k, the votes are aggregated, and the most likely physiological state for patient k is selected (step 814). This process is repeated for the patients in the testing database until the testing process is complete (step 820).

At step 802, three iteration parameters n, j, and k are each initialized to one. The iteration parameter n is representative of a classifier, the iteration parameter j is representative of a respiratory cycle, and the iteration parameter k is representative of a patient.

At step 804, the n^{th} classifier votes on the j^{th} respiratory cycle of the k^{th} patient in the testing data set. For example, the decision rule of a classifier may indicate that a particular feature perfectly discriminates between two physiological states. In this case, the classifier may use the same feature on the testing dataset to cast its vote. In general, the decision rule of the n^{th} classifier is applied to one or more extracted features of the j^{th} respiratory cycle of the k^{th} patient.

At decision block 806, it is determined whether the iteration parameter n equals the desired total number of classifiers N. If not, the iteration parameter is incremented at step 808, and the process returns to step 804 for the next classifier to vote.

At decision block 810, it is determined whether the iteration parameter j equals the desired total number of respiratory cycles J to evaluate in a patient. If not, the iteration parameter j is incremented and the iteration parameter n is reinitialized to one at step 812, and the process returns to step 804 for the 1^{st} classifier to vote on the next respiratory cycle for the k^{th} patient.

When both iteration parameter n and j reach their final values, the vote aggregator 436 aggregates the votes across the N classifiers and across the J respiratory cycles to select the most likely physiological state for patient k at step 814. As an example, the vote aggregation may be performed in two stages. For example, each of the N classifiers has voted on each of the J respiratory cycles, classifying each respiratory cycles as CHF or COPD, for example. In one embodiment, the votes across the N classifiers are aggregated for each respiratory cycle, resulting in J votes (one for each respiratory cycle). Then, the J votes may be aggregated. In an example, to aggregate votes, the majority (or some other fraction) of the votes may be selected. In an example, N=50 and J=35. Each classifier votes on each of the 35 first valid breaths in the new test record, classifying each breath as CHF or COPD. Whatever the majority (or some other selected fraction) of the verdicts comes out to be, out of the 35 votes, is the determination by that classifier as to whether that record (as opposed to each individual breath) belongs to a CHF or COPD patient.

After the voting process for the first patient in the testing group has been completed, the iteration parameters n and j are both reinitialized to one, and the iteration parameter k is incremented at step 818. This voting process is repeated for the remaining patients until testing is complete at step 820, when the iteration parameter k reaches K at decision block 816.

FIG. 9 is a flowchart of a method used by the application stage 206 to apply validated classifiers (i.e., received from the testing stage 204) to a patient's capnogram data and to identify a predicted physiological state of the patient. The method 900 includes the steps of receiving a capnogram (step 902), preprocessing the capnogram (step 904), and developing a record template (step 906). If it is desirable to display the template (decision block 908), the template view is displayed (step 910). Otherwise, outlier data are identified and removed (step 912), and waveform features are extracted from the capnogram (step 914). Then, the classifiers vote on the capnogram (step 916). Depending on a desired display, a histogram view is displayed (step 920), and/or a highlighted waveform view is displayed (step 922).

At step 902, a the receiver 552 receives a patient's data including a capnogram. In particular, the capnogram may be a set of discrete sample values corresponding to concentrations of CO₂ of exhaled breath of a patient and may include multiple respiratory cycles from the same patient. As an example, a computing device such as the computing device 600 in FIG. 6 may receive the capnogram over a network or over any communications channel.

At step 904, the preprocessor 554 processes the capnogram. As an example, the capnogram may be preprocessed to get the capnogram into a certain form, such as by controlling the amplitude of the capnogram or adjusting other characteristics of the capnogram. For example, the capnogram may be quantized, filtered, smoothed, downsampled, upsampled, interpolated, or any number of other processing techniques may be performed on the capnogram.

At step 906, a record template is developed. As an example, the capnogram may be divided into segments corresponding to the respiratory cycles in the capnogram. The segments may be combined to form a representative respiratory cycle. The combination may be by first aligning all the segments so they pass through a given level of exhaled CO₂ concentration at a fixed time after the initial time of the template, and then computing one or more statistics of the segments, such as a mean, median, standard deviation, interquartile range, or any other suitable statistic. The record template may be the representative respiratory cycle.

At decision block 908, it is determined whether it is desirable to display the record template. For example, a user at a user device may provide user input indicative of a desire to display the record template. In this case, the template view is displayed at step 910. Example template views are shown in FIGS. 10 and 11. In addition to displaying the waveform of the representative respiratory cycle, the template view may further include suitable statistics, such as standard deviation or interquartile ranges to indicate variability across different respiratory cycles in the capnogram. An example template view with standard deviation bars is shown in FIG. 6.

At step 912, the feature extractor 555 extracts one or more waveform features of the capnogram. For example, waveform features may include the duration of exhalation, duration of the respiratory cycle, signal amplitude at the end of exhalation, slope at the beginning of exhalation, slope at the end of exhalation, curvature at particular stages of exhalation, or any other suitable feature of a capnogram. Examples of features are described in more detail in relation to FIGS. 13-15.

At step 914, the outlier remover 556 identifies and removes outlier data, which may correspond to outlier exhalations. In particular, portions of the capnogram may be identified as anomalous and may be discarded. For example, anomalous portions may be identified if the corresponding sample values fall outside a range. A suitable range may be defined by a number of standard deviations around the representative respiratory cycle. An example display of excluded data in a capnogram is shown in FIG. 12.

At step 916, the classifiers vote on the capnogram received at step 902. In particular, the capnogram received at step 902 may correspond to a patient with an unknown physiological state, and it is desirable to use the systems and methods described herein to determine the likelihoods of various physiological states for the patient. The vote aggregator 558 then aggregates the votes to determine the likelihoods of the candidate physiological states of the patient. The voting collection and aggregation process is described in more detail in relation to FIG. 8, and may include the steps of 802-814 to determine a most likely physiological state for the patient. The likelihoods and/or most likely physiological state for the patient may be displayed to a user such as a clinician over the display renderer 562 within the user interface 560. In addition, the display renderer 562 may also display a confidence score representative of a confidence in the predicted diagnosis.

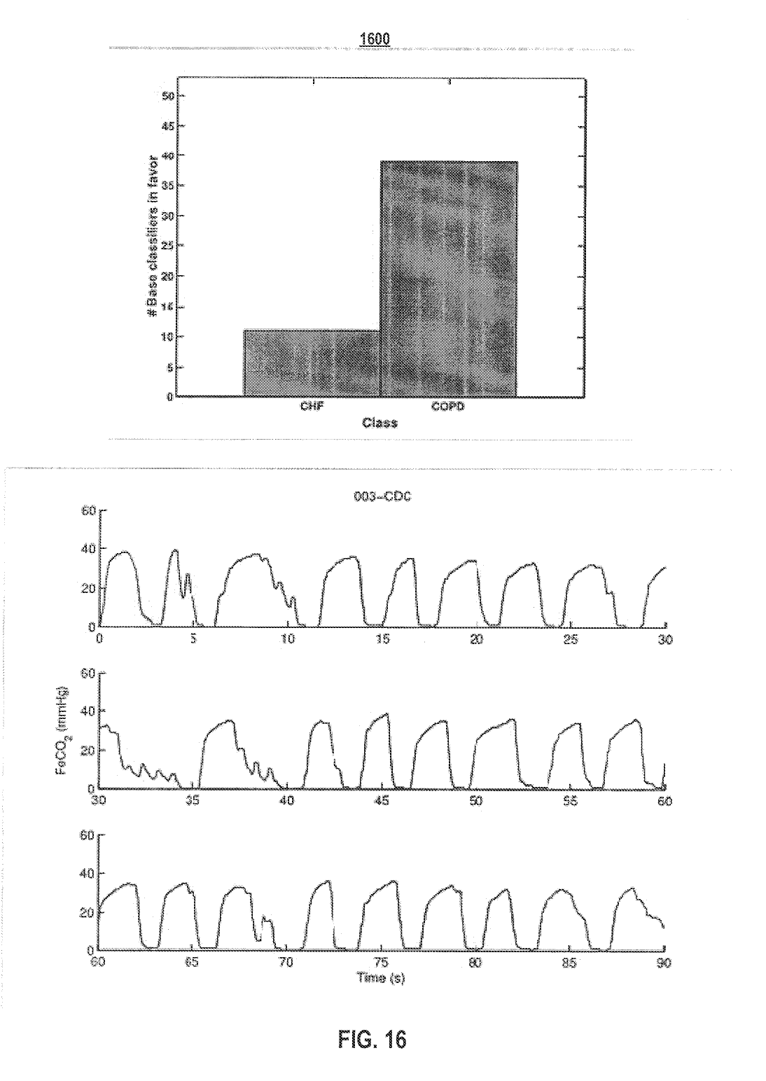

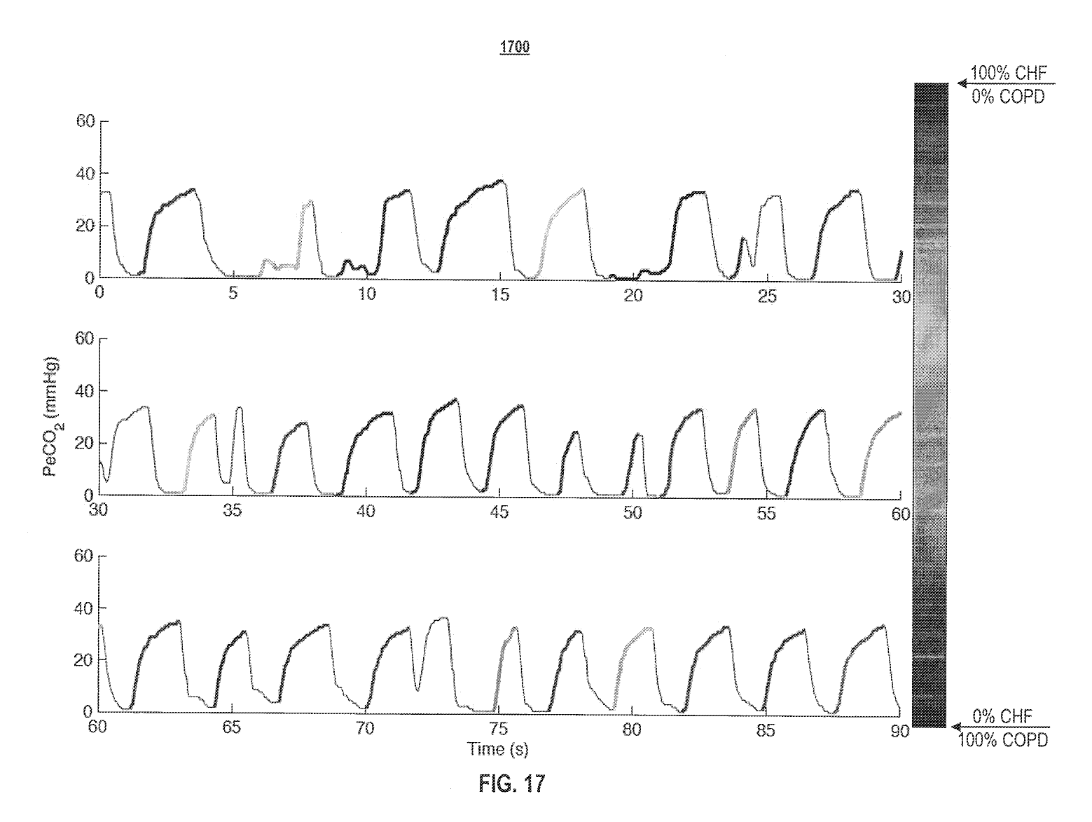

At decision block 918, it is determined whether it is desirable to display a histogram of the voting results. For example, a user at a user device may provide user input indicative of a desire to display the histogram. Depending on the user's selection, a histogram view is displayed at step 920 (an example histogram is shown in FIG. 16). Similarly, at decision block 919, it is determined whether it is desirable to display a highlighted waveform view. For example, the user at the user device may provide user input indicative of a desire to display the highlighted waveform view. Depending on the user's selection, a highlighted waveform view is displayed at step 922 (an example highlighted waveform view is shown in FIG. 17).

FIGS. 16 and 17 are example displays of results from the disease classification process, according to an illustrative embodiment. In particular, systems and methods described herein may include a user interface for interacting with a user (such as a doctor, nurse, or trained professional, for example). As an example, the user interface may include a touch screen monitor in a hospital. The user may provide user input over the user interface, such as any number of parameters of the systems and methods described herein.

Examples of user input include a capnogram, a patient, a plurality of respiratory cycles, a number of classifiers, a number of respiratory cycles, feature values associated with the classifiers, the plurality of candidate physiological states, and likelihood threshold values. For example, the user may select to characterize the likelihoods of different physiological states of a patient. The user may provide as input to the user interface an indication of the patient, such as the patient's capnogram or any other identifying patient data. The user may then select one or more respiratory cycles of the patient's capnogram, or the systems and methods described herein may automatically select one or more respiratory cycles of the capnogram to analyze. The user may then also provide input such as the total number of desired classifiers or a total number of respiratory cycles to use. As another example, the user may provide statistical parameters. For example, the user may indicate that it is desirable to only analyze the data if the representative respiratory cycle has low variance. In this case, the user may indicate a threshold multiple of the standard deviation, for example, where the number of respiratory cycles is increased until the representative respiratory cycle satisfies the threshold conditions set by the user. In addition, the user may also provide a list of candidate physiological states, whose likelihoods will be compared. As an example, the user interface may display a list of physiological states, and the user may select one or more of the physiological states from the list. The user may also select likelihood threshold values for the selected physiological states. For example, the user interface may display the physiological state determined to be most likely after the analysis is complete. In another example, the user interface may be configured to only display the most likely physiological state if the associated likelihood exceeds the threshold.

In some embodiments, the user interface includes displaying the vote in a histogram view (as shown in FIG. 16). The results of the classification process are viewed as a histogram revealing how many base classifiers voted in favor of each possible choice. This histogram may be displayed over different windows on the capnogram and reveals some measure of confidence in the classification.

In some embodiments, the user interface includes displaying a color-coded waveform in which the breakdown of each exhalation's votes is revealed by the color of the plotted exhalation (as shown in FIG. 17). The results of the classification process may be viewed as a highlighted waveform whose specific color represents the percentage of classifier votes for a particular disease state at any given time window. The color could gradually change with the number of base classifiers voting in a given direction. In this way, the classification of each exhalation is graphically displayed.

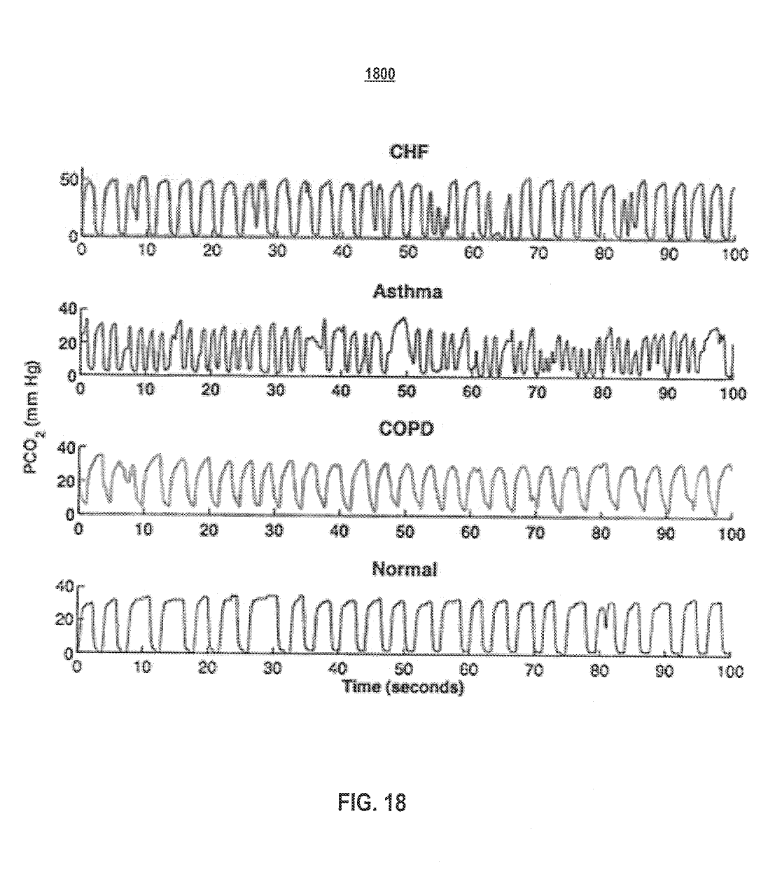

FIG. 18 is a diagram of example capnograms from patients with various physiological states. In particular, the capnogram shapes vary greatly from patients with different diagnoses. The systems and methods described herein allow for automatic disease classification using machine learning techniques as applied to capnogram data. In particular, features are extracted from capnograms recorded from patients in a training data set. The patients in the training data set have known physiological states, and in a training stage, classifiers are trained on the extracted features and the known states. Then, in a testing stage, the trained classifiers are validated on a testing data set, and the resulting predicted physiological states are compared to the known states of the patients in the testing group. Finally, in an application stage, the validated classifiers are applied to a patient's capnogram data to determine the likelihoods of various physiological states and/or the most likely physiological state of the patient.

While various embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. An automated method to quantitatively identify a patient's physiological state based on one or more capnograms, comprising:
acquiring one or more capnograms, each capnogram being associated with a patient and including one or more respiratory cycles;
extracting one or more features from the one or more respiratory cycles;
providing one or more classifiers based on the one or more extracted features;
selecting, with each classifier, a physiological state from one or more candidate physiological states for each of the one or more respiratory cycles;
determining a likelihood value for each of the selected physiological states; and
identifying a physiological state of the patient based on the likelihood values.

2. The method of claim 1, wherein the patient's physiological state pertains to a cardiorespiratory condition.

3. The method of claim 2, wherein the cardiorespiratory condition includes one or more of congestive heart failure, asthma, bronchiolitis, cystic fibrosis, bronchopulmonary dysplasia, chronic obstructive pulmonary disease, or normal.

4. The method of claim 1, wherein the acquiring includes detecting one or more of a time of an onset of a relevant segment of a respiratory cycle, an amplitude of an onset of a relevant segment of a respiratory cycle, a time of an end of a relevant segment of a respiratory cycle, and an amplitude of an end of a relevant segment of a respiratory cycle.

5. The method of claim 1, wherein the acquiring includes segmenting a capnogram into a plurality of respiratory cycles.

6. The method of claim 1, wherein the acquiring includes constructing a template of a representative respiratory cycle for one or more respiratory cycles in a capnogram and constructing the template includes computing one or more of a mean, median, standard deviation, or interquartile range at selected time points during the respiratory cycle, further comprising identifying an outlier portion of the capnogram based on the template.

7. The method of claim 1, wherein extracting the one or more features includes fitting a portion of the capnogram to a parameterized function.

8. The method of claim 1, wherein the one or more feature values are based on physiologically significant parameters, and a feature value is selected from the group consisting of a duration of exhalation, a duration of the respiratory cycle, respiratory rate, a signal amplitude at the end of exhalation, a slope at the beginning of exhalation, a slope at the end of exhalation, a measure of curvature at the beginning of exhalation, a measure of curvature at an intermediate point of exhalation, and a measure of curvature at the end of exhalation.

9. The method of claim 1, wherein the one or more classifiers are trained using a machine learning method.

10. The method of claim 1, wherein selecting a physiological state is performed by voting.

11. The method of claim 1, wherein identifying the patient's physiological state includes comparing the likelihood values.

12. A system for quantitatively identifying a patient's physiological state based on one or more capnograms, comprising a processor configured to:
acquire one or more capnograms, each capnogram being associated with a patient and including one or more respiratory cycles;
extract one or more features from the one or more respiratory cycles;
provide one or more classifiers based on the one or more extracted features;
select, with each classifier, a physiological state from one or more candidate physiological states for each of the one or more respiratory cycles;
determine a likelihood value for each of the selected physiological states; and
identify a physiological state of the patient based on the likelihood values.

13. The system of claim 12, wherein the processor is further configured to display the capnogram and the classification results.

14. The system of claim 13, wherein the processor performs in real time one or more of the acquiring, extracting, providing, selecting, determining, identifying, and displaying.

15. The system of claim 13, wherein the processor displays one or more of the recorded capnogram, related capnogram parameters, the computationally processed capnogram, a representative template, the classification results, and the likelihood values.

16. The system of claim 15, wherein the related capnogram parameters are one or more of end-tidal carbon dioxide level, exhalation duration, and respiratory rate.

17. The system of claim 15, wherein the processor displays one or more of a highlighted or color-coded segment of a respiratory cycle, an identification of an outlier cycle, and an identification of intervals of poor signal quality.

18. The system of claim 15, wherein the processor displays one or more respiratory cycles superposed on the template.

19. The system of claim 15, wherein the processor displays the likelihood values by a representation of the classifier votes.

20. A system for determining a physiological state associated with a capnogram, comprising:
a receiver configured to receive a plurality of capnograms, wherein each capnogram includes a plurality of respiratory cycles and is associated with a patient and one or more feature values;
a processor configured to:
separate the plurality of capnograms into a training set and a testing set, wherein each capnogram in the training set is associated with a physiological state;
generate a plurality of classifiers, wherein each classifier is based on the associated feature values and the physiological states of a subset of capnograms in the training set;
select a capnogram from the testing set;
identify, with each classifier, a candidate physiological state in a plurality of candidate physiological states for each respiratory cycle in the identified capnogram;
aggregate the candidate physiological states across the classifiers to generate an elected physiological state for each respiratory cycle in the identified capnogram; and
aggregate the elected physiological states across the respiratory cycles to determine the likelihood of a physiological state of the patient.
